(19) United States
(12) Patent Application Publication
(10) Pub. No.: US 2026/0363662 A1
(43) Pub. Date: Nov. 19, 2026

(54) ULTRASONIC CURVED LINEAR ARRAY OPERABLE IN THE CONTINUOUS WAVE MODE

(71) Applicant: KONINKLIJKE PHILIPS N.V., EINDHOVEN (NL)

(72) Inventors: Changhong Hu, Bothell, WA (US); Andrew Lee Robinson, Ferndale, WA (US); Steven Russell Freeman, Seattle, WA (US); Matthew Robert Rielly, Seattle, WA (US); Wei Lu, Mill Creek, WA (US)

(21) Appl. No.: 19/514,959

(22) PCT Filed: Aug. 20, 2024
(86) PCT No.: PCT/EP2024/073242

§ 371 Date: Feb. 27, 2026

Related U.S. Application Data
(60) Provisional application No. 63/535,148, filed on Aug. 29, 2023.

(51) Int. Cl. A61B 8/00; A61B 8/06

(52) U.S. Cl. CPC A61B 8/4483; A61B 8/06; A61B 8/488

(57) ABSTRACT

Continuous wave (CW) Doppler analysis is performed using an ultrasound system with a linear array transducer probe. The linear array may be curved or flat. The array transducer has more transducer elements than the number of channels of the beamformer of the ultrasound system. To accommodate this disparity, some of the elements of the array are multiplexed to the beamformer so that only one of a pair of elements is coupled to the beamformer at any one time. A plurality of the elements of the array transducer are not multiplexed and are coupled directly to the beamformer. During imaging both multiplexed and unmultiplexed transducer elements can be used to produce signals for an image, and during CW Doppler analysis only unmultiplexed transducer elements are used for the CW Doppler transmit and receive apertures.

This invention relates to ultrasound imaging systems and, in particular, to an ultrasound system with a curved linear array probe operable in the continuous wave Doppler mode and other imaging modes.

Diagnostic ultrasound can be used to image the anatomy of the body by producing two and three dimensional anatomical images. It can also be used to measure and display characteristics of blood flow using Doppler techniques. Flow velocity is a function of the phase shift between the transmitted and received ultrasound waves. Doppler information can be displayed anatomically such as with colorflow imaging, and spectrally with a scrolling spectrum of flow velocities. The common technique used for colorflow imaging is pulsed wave Doppler, whereby flow is sampled periodically and repeatedly at locations where blood flow is occurring. The samples acquired over time from each location in an image field, referred to as an ensemble, are processed by Fourier transform processing to estimate flow characteristics occurring over time at each location. These flow measurements are then converted into color values for display anatomically in conjunction with an anatomical image, with different shades of color depicting different blood flow velocities, for instance. The pulsed Doppler flow measurements from a selected anatomical location, referred to as a sample volume, can also be displayed as a continuously scrolling spectrum of flow as a function of time, depicting peak and minimum flow velocities during each heart cycle, for example.

Another Doppler technique for measuring blood flow is continuous wave (CW) Doppler. Unlike pulsed Doppler, the CW Doppler signal is transmitted and received continuously. Transmission is done from one aperture of the probe transducer, and reception is done from another aperture of the transducer. A disadvantage of CW Doppler is that, while pulsed Doppler provides range information from the time-of-flight of the ultrasound pulse to and from the target region, such ranging is not possible when the Doppler wave is continuously transmitted and received. The advantage of CW Doppler is its ability to measure high flow velocities and velocity changes without ambiguity, as it does not suffer from aliasing as pulsed wave Doppler does when the pulse rate does not satisfy the Nyquist sampling criterion for the high flow velocities present at the target. CW Doppler thus finds great utility for conditions involving stenotic and regurgitant flow which are characterized by high flow velocities in the range of meters per second.

Different types of ultrasound probes, e. g., linear arrays, phased arrays, etc. are used to image and diagnose conditions in different regions of the body. An ultrasound probe which is widely used for abdominal and obstetric imaging is the curved linear array probe, in which the array elements are arranged in an outwardly convex arc rather than a straight (flat) line. The curvature of the array provides some of the beam steering physically rather than entirely by phasing of the elements, reducing electronic delay requirements for the ultrasound system and providing greater field of view, including at the skin line. Since abdominal and obstetrical imaging are generally performed at greater depths in the body than other, more peripheral, exams, it is desirable to reduce image noise factors as much as possible so that clearer image are produced from the ultrasound signals attenuated by the greater depths of travel. One way to reduce image noise, or clutter, is to reduce the ultrasound beam sidelobe clutter by reducing the magnitude of transducer sidelobes. This may be done by employing a greater number of smaller elements in the array, with the smaller pitch of the elements providing the desired sidelobe reduction. This poses a problem when it is desirable to increase the number of transducer elements to a number which is greater than the number of channels of the ultrasound system beamformer. An approach which enables this dichotomy is to multiplex the transducer elements, so that only a selectable subset of the full number of transducer elements is connected to the system beamformer at any point in time. This poses a dilemma for the ultrasound system designer in how to configure the multiplexing to enable effective CW Doppler diagnosis and other scanning modes in an ultrasound system in which the number of channels of the beamformer is less than the number of elements of the array transducer.

In accordance with the principles of the present invention, an ultrasonic diagnostic imaging system with a linear array probe having a number of transducer elements greater than the number of channels of the system beamformer, and capable of performing CW Doppler analysis, is described. The linear array probe, which may be curved or flat, contains a plurality of multiplexers which selectably couple certain ones of the elements of the array to the ultrasound system and hence to the channels of the system beamformer. While some of the elements of the array transducer are multiplexed, others are not multiplexed. transducer elements which are not multiplexed are used for the transmit aperture and for the receive aperture when the array probe is operated to perform CW Doppler analysis. In other modes of the probe, both multiplexed and unmultiplexed elements can be used for imaging.

A preferred ultrasound system for performing CW Doppler analysis comprises a linear array transducer probe having a given number of transducer elements arranged in a curved arc. Some of the transducer elements are multiplexed to single multiplexer terminals and others of the transducer elements are not multiplexed. A system beamformer has a number of beamformer channels which is less that the given number of transducer elements of the curved arc of elements of the curved array probe. A B mode image processor is coupled to the system beamformer and a Doppler processor is coupled to the system beamformer. A display processor is coupled to the B mode image processor and the Doppler processor and is adapted to produce a B mode image and/or a CW spectral Doppler image for display. A display is coupled to the display processor. The B mode image is produced from signals produced by the curved array probe using at least some of the multiplexed transducer elements and the CW spectral Doppler image is produced from signals produced by the curved array probe using elements that are not multiplexed.

A method of the present invention for performing CW Doppler analysis comprises scanning a target anatomy with a linear array transducer probe to acquire a B mode image, the array transducer comprising a plurality of transducer elements which are multiplexed and a plurality of transducer elements which are not multiplexed, multiplexed and unmultiplexed elements being coupled to a beamformer with fewer channels than the number of transducer elements of the transducer array. A Doppler line is adjusted over the image to intersect a target in the B mode image. A sample volume cursor is adjusted over the target in the B mode image. CW Doppler signal acquisition is started using elements of the array transducer which are not multiplexed.

In the drawings:

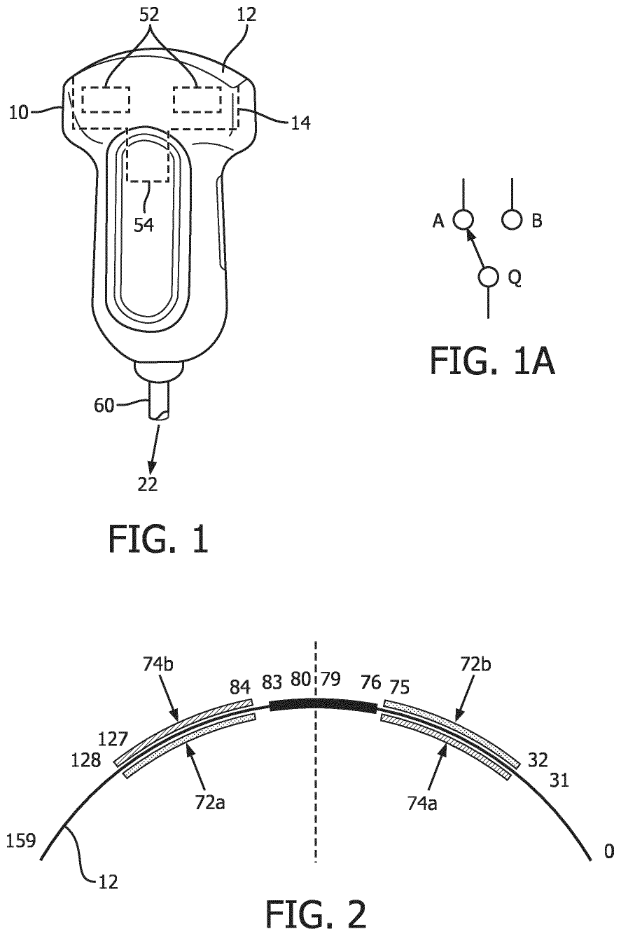

FIG. 1 illustrates a curved array ultrasound probe constructed in accordance with the principles of the present invention.

FIG. 1a schematically illustrates the operation of the multiplexers of the probe of FIG. 1.

FIG. 2 illustrates the elements of a curved array transducer of the present invention and their apertures for CW Doppler use.

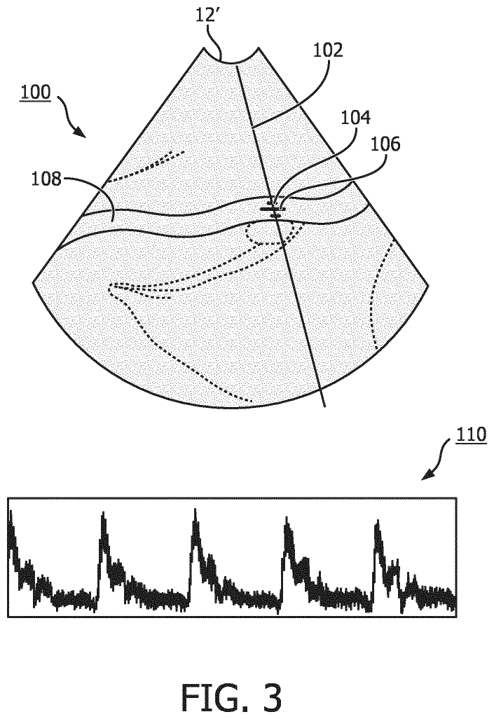

FIG. 3 illustrates the display of an ultrasound system of the present invention, including a B mode image and a scrolling Doppler display.

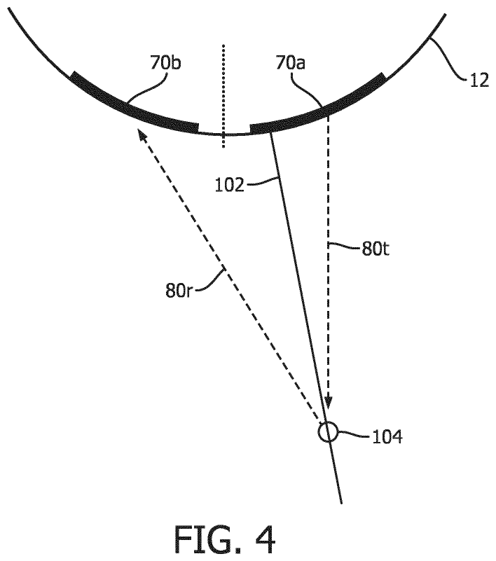

FIG. 4 illustrates the beams transmitted and received from a curved array transducer of the present invention and the Doppler line shown on the ultrasound system display for a CW Doppler procedure.

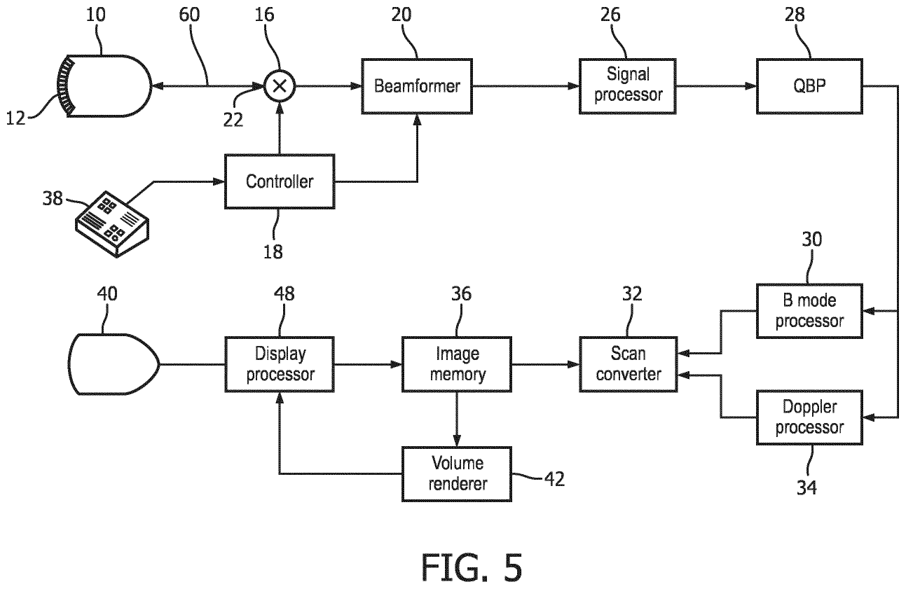

FIG. 5 illustrates in block diagram form an ultrasound system configured in accordance with the principles of the present invention.

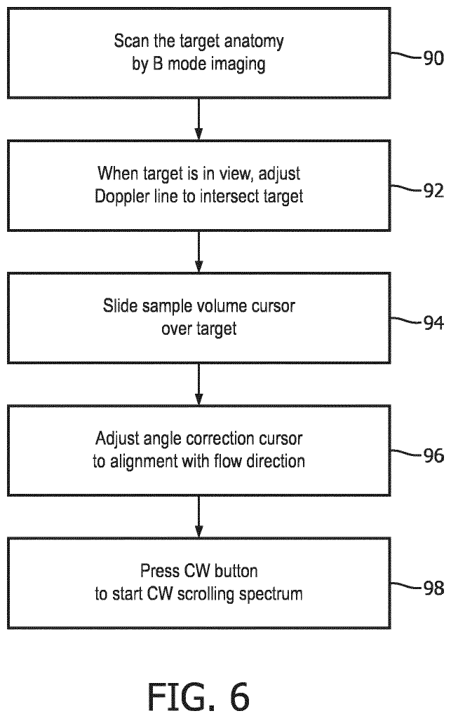

FIG. 6 illustrates a method for performing imaging-guided CW Doppler measurement in accordance with the principles of the present invention.

Referring first to FIG. 1, a curved array ultrasound probe 10 is shown. In use, the probe is held by the handle (bottom) portion and the curved array transducer 12, which is covered by a polymeric lens material, is pressed against the body of the subject during scanning. The curved array transducer is generally coupled to a mating backing in the form of a printed circuit board (PCB) 14 to which the array elements are electrically connected. Electrical connections may alternatively be made to a flex circuit behind the array. A plurality of multiplexer integrated circuits 52 are located on the PCB or flex circuit. The number of multiplexer IC modules needed depends upon the packaging density of the modules and the number of elements of the curved array which are multiplexed. A schematic of a multiplexer is shown in FIG. 1a, where it is seen that the switch arm of the multiplexer, shown as an arrow, can be set to couple the transducer element coupled to terminal A or alternatively the transducer element coupled to terminal B to the common terminal Q. The common Q terminals of multiplexers 52 are coupled to channels of the system beamformer by way of the probe cable 60, and the A and B terminals of the multiplexers are coupled to elements of the curved array transducer. Elements of the curved array which are not multiplexed are coupled to channels of the beamformer by means of conductive traces on the PCB or flex circuit and conductors of the cable 60. The probe cable is connected to the PCB 14 by a connector at the proximal end 54 of the PCB.

FIG. 2 illustrates how the elements of a curved array transducer are used for scanning in a preferred embodiment of the present invention. The illustrated curved array transducer has 160 elements, numbered sequentially from right to left along the curved array, starting with element 0 and ending with element 159. A vertical dashed line demarcates the center of the array. when operating in the CW Doppler mode, two apertures of the array are used, one for transmission and the other for reception. One aperture is formed of elements 32 to 75, and the other aperture is formed of elements 84 to 127. The assignment of the CW apertures for transmission or reception depends upon which side of the array (to the left or right of the dashed center line) the target for CW analysis is located. If the target is located to the left of center, the aperture formed by elements 84-127 is used for transmission as indicated by arrow 72a, and the aperture formed by elements 32-75 is used for reception as indicated by arrow 72b.

When the target is to the right of center, aperture assignment for transmission and reception is reversed, as indicated by transmit arrow 74a and reception arrow 74b. Thus transmission is done from the aperture on the side of the array where the target is located.

Elements 76-83 in the center of the array are not active during CW scanning. They are used to provide isolation between the CW apertures on either side of these center elements. The elements 0-31 and 128-159 are not used during CW scanning.

In accordance with the principles of the present invention, the array elements used for the CW apertures, 32-75 and 84-127 in the example of FIG. 2, are coupled directly to conductors of the probe cable 60 and thence to channels of the system beamformer; they are not coupled to the beamformer by multiplexers. Since the total number of elements used for the CW transmit and receive apertures, 88 in this example, is less than the number of channels of the system beamformer, 128 channels, there is no need to employ multiplexers when performing CW Doppler scanning.

During other scanning modes when the full array of 160 elements may be used, the multiplexers 52 are used to couple the necessary elements to the 128 channel system beamformer. For example, when scanning a B mode image, 100 elements may be used to scan each line of the B mode image. Elements 0-99 are used to produce the first line on the right, then elements 1-100 are used to form the next adjacent line, then element 2-101 are used to form the third line, and so on across the array. A suitable multiplexer configuration for a linear array probe operating in this manner would be to multiplex only elements 0-31 and 120-159, with the other elements directly coupled to beamformer channels. The multiplexers and elements are arranged so that no two elements that operate in an aperture at the same time are coupled to the same multiplexer. A suitable coupling for the CW and B mode scanning just described would be to couple elements 0 and 128 to a first multiplexer, elements 1 and 129 to a second multiplexer, elements 2 and 130 to a third multiplexer, and so on. The scanning sequence of 100 adjacent array elements for each B mode line can then be performed with no conflicts in multiplexer operation.

FIG. 3 illustrates a display for a curved array ultrasound system which performs both B mode imaging and Doppler flow analysis. A B mode sector display 100 shows anatomy with a blood vessel 108 passing through it. The image is produced by a curved array transducer 12′ positioned at the top of the image during scanning. The sonographer positions a Doppler line 102 over the image which intersects the blood vessel at a desired target location. The doppler line can be moved left and right, extending from the position 12′ of the curved array. The sonographer then moves a sample volume cursor 104 until it is positioned over the point in the anatomy where blood flow is to be assessed. A flow cursor 106 is then positioned in line with the blood flow direction, generally by tilting it until it is parallel to the walls of the blood vessel at the sample volume. The illustrated set of image cursors can be used for both CW Doppler and pulsed Doppler spectral scanning.

Once this preparatory alignment has been completed, the sonographer presses the “CW” button on the control panel of the ultrasound system to start CW Doppler operation. The B mode image 100 is then frozen on the display and the CW Doppler display 110 begins to scroll across the screen. While the Doppler line may give the impression that the Doppler beam is transmitted down the Doppler line 102 to the sample volume with reflected waves returning back to the transducer along the line, that is not what is actually happening when separate transmit and receive apertures are used. FIG. 4 illustrates a more accurate representation of what happens during scanning. In this example the sample volume 104 is to the right of the center of the curved array 12, so the CW aperture 70a on the right is used for transmission. CW Doppler waves are continuously transmitted and phase-steered so that the beam center is directed as shown by dashed arrow 80t. Some of the reflected Doppler waves are returned toward the receive aperture 70b on the left side of the curved array 12, and receive beam steering is done along a beam center 80r. Doppler waves are continuously transmitted from the CW aperture 70a and continuously received by the CW aperture 70b during CW Doppler operation.

Referring now to FIG. 5, an ultrasonic diagnostic imaging system constructed in accordance with the principles of the present invention is shown in block diagram form. A linear array transducer 12, illustrated here as a curved linear array, is provided in an ultrasound probe 10 for transmitting ultrasonic waves and receiving echo information. The transducer array 12 may be a one- or two-dimensional array of transducer elements capable of scanning in two or three dimensions, for instance, in both elevation (in 3D) and azimuth. The probe 10 contains multiplexers 52 as shown in FIG. 1 which multiplex the coupling of pairs of some of the elements of the array to the system beamformer while other elements, including those of the CW Doppler apertures, are not multiplexed. Elements of the transducer array 12 and the multiplexers are coupled to a system beamformer 20 by means of the probe cable 60 and probe connector 22 on the end of the cable, and may be preceded in the probe by an optional microbeamformer which controls transmission and reception of signals by the array elements. Microbeamformers are capable of at least partial beamforming of the signals received by groups or “patches” of transducer elements as described in U.S. Pat. No. 5,997,479 (Savord et al.) , U.S. Pat. No. 6,013,032 (Savord), and U.S. Pat. No. 6,623,432 (Powers et al.) The main system beamformer 20 is coupled to the probe cable by a transmit/receive (T/R) switch 16 which switches between transmission and reception. The transmission of ultrasonic beams from the transducer array 12, the reception of echo signals by the system beamformer 20, and the operation of the multiplexers 52 is under control of a controller 18, which receives input from the user's operation of the user interface or control panel 38. Among the transmit characteristics controlled by the transmit controller are the number, spacing, amplitude, phase, frequency, polarity, and diversity of transmit waveforms. Beams formed in the direction of pulse transmission may be steered straight ahead from the transducer array, or at different angles on either side of an unsteered beam direction for a wider sector field of view. For some applications, unfocused plane waves may be used for transmission, and in this case the receive beams may interrogate the entire field of view simultaneously. Most 1D array probes of relatively small array length, e.g., a 128-element array, do not use a microbeamformer but are driven from and respond directly to the main beamformer.

The echoes received by transducer elements of the array during both imaging and CW Doppler operation are beamformed by appropriately delaying them and then combining them in the system beamformer 20 under direction of the controller 18. The coherent echo signals undergo signal processing by a signal processor 26, which includes filtering by a digital filter and noise reduction as by spatial or frequency compounding. The filtered echo signals are coupled to a quadrature bandpass filter (QBP) 28. The QBP performs three functions: band limiting the RF echo signal data, producing in-phase and quadrature pairs (I and Q) of echo signal data, and decimating the digital sample rate. The QBP comprises two separate filters, one producing in-phase samples and the other producing quadrature samples, with each filter being formed by a plurality of multiplier-accumulators (MACs) implementing an FIR filter. The signal processor can also shift the frequency band to a lower or baseband frequency range, as can the QBP. The digital filter of the signal processor 26 can be a filter of the type disclosed in U.S. Patent No. 5,833,613 (Averkiou et al.), for example.

The beamformed and processed coherent echo signals are coupled to a B mode processor 30 which produces a B mode image of structure in the body such as tissue. The B mode processor performs amplitude (envelope) detection of quadrature demodulated I and Q signal components by calculating the echo signal amplitude in the form of (I²+Q²)^{1/2}. The quadrature echo signal components are also coupled to a Doppler processor 34. The Doppler processor 34 estimates the phase shift between the transmitted and received Doppler signals at and in the vicinity of the sample volume with a phase comparator or, for pulsed Doppler, a fast Fourier transform (FFT) processor. The Doppler shift is proportional to flow velocity at the sample volume, including rapid jets emanating from a stenosis or regurgitant valve. For a color Doppler image, the estimated Doppler flow values at each point in a blood vessel are wall filtered and converted to color values using a look-up table. The wall filter has an adjustable cutoff frequency above or below which motion will be rejected such as the low frequency motion of the wall of a blood vessel when imaging flowing blood. The B mode image signals and the pulsed Doppler flow values are coupled to a scan converter 32 which converts the B mode and Doppler samples from their acquired R-θ coordinates to Cartesian (x, y) coordinates for display in a desired display format, e.g., a rectilinear display format or a sector display format for an anatomical image, or a scrolling display of consecutively received and processed Doppler values for a spectral display. For an anatomical image, either the B mode image or the Doppler image may be displayed alone, or the two shown together in anatomical registration in which the color Doppler overlay shows the blood flow in tissue and vessels in the image as shown in FIG. 3. Another display possibility is to display side-by-side images of the same anatomy which have been processed differently. This display format is useful when comparing images. The spectral Doppler display may also be shown alone or in conjunction with an anatomical image.

The spectral and image data produced by the B mode processor 30 and the Doppler processor 34 are coupled to an image data memory 36, where it is stored in memory locations addressable in accordance with the spatial locations from which the image values were acquired. Spectral Doppler data is stored in accordance with the time at which it was acquired. Image data from 3D scanning can be accessed by a volume renderer 42, which converts the echo signals of a 3D data set into a projected 3D image as viewed from a given reference point as described in U.S. Pat. No. 6,530,885 (Entrekin et al.) The 3D images produced by the volume renderer 42 and 2D images produced by the scan converter 32 as well as the data for lines of a spectral display are coupled to a display processor 48 for further enhancement, buffering and temporary storage for display on an image display 40. The display processor also includes a graphics generator for producing lines and graphics such as the cursors shown in FIG. 3 for display in conjunction with the ultrasound images.

A method for performing CW Doppler analysis of a subject is illustrated in FIG. 6. At the starting step 90, the anatomy of the subject is scanned with a linear array transducer probe to image the target region. Preferably the linear array transducer is a curved array having a large number of finely pitched elements, such as 160 or 192 elements. The probe may contain a single curved row of transducer elements, or multiple adjacent rows for operation in the elevation dimension. Coherent image values are produced by a system beamformer for display. When elements of the curved array are multiplexed, the system beamformer can have fewer channels than the number of transducer elements of the array, such as a standard 128-channel beamformer. Both multiplexed and unmultiplexed elements can be used for imaging. When the target is in view in step 92, the sonographer adjusts the Doppler line over the image so that it intersects the anatomical target, such as a blood vessel or heart valve. The sonographer then adjusts a sample volume cursor to be located over the target by sliding it along the Doppler line in step 94. In step 96 the sonographer adjusts an angle correction cursor so that it is aligned with the flow direction so that Doppler values can be corrected in consideration of the angle between the direction of flow and the beam direction. Once the target is clearly in view and has been targeted as described above, the “CW” button on the ultrasound system is pressed in step 98 to start CW Doppler acquisition and production of a scrolling spectral display.

Since the anatomical image is frozen on the ultrasound display during CW Doppler acquisition, it is important for the sonographer to hold the array probe steady so that the beams remain on target. Should probe or patient movement cause loss of target acquisition, the sonographer can press the “Image” button on the ultrasound system to return to the imaging mode so that the probe can be re-aimed to again acquire the target and reset the graphics over the image.

Although the aforementioned method recites steps that may be performed by a sonographer or other user, the scope of the method also contemplates that one or more of the steps 90-98 may be performed automatically under control of a system computer processor and software, such as image recognition software enabled by pre-trained models. The target location for CW Doppler analysis can be continually tracked and targeted by means of speckle-tracking.

The concepts of the present invention may be applied in various ways, depending upon the objectives of the ultrasound designer. As mentioned above, the linear array transducer may be a curved linear array or a flat linear array. The curved linear array provides the benefits of a greater field of view and reduced steering delays. While the example of FIG. 1 illustrates the multiplexers 52 located in the transducer housing of the probe, the multiplexers may alternatively be located in the case of the connector 22 at the other end of the cable. Other variations will readily occur to those skilled in the art of ultrasound design.

It should be noted that an ultrasound system suitable for use in an implementation of the present invention, and in particular the component structure of the ultrasound system of FIG. 5, may be implemented in hardware, software or a combination thereof. The various embodiments and/or components of an ultrasound system, for example, the beamformer, the signal processor, the B mode processor, and the Doppler processor, or components, processors, and controllers therein, also may be implemented as part of one or more computers or microprocessors. The computer or processor may include a computing device, an input device, a display unit and an interface, for example, for accessing the Internet. The computer or processor may include a microprocessor. The microprocessor may be connected to a communication bus, for example, to access a PACS system or the data network for importing images. The computer or processor may also include a memory. The computer or processor further may include a storage device, which may be a hard disk drive or a removable storage drive such as a floppy disk drive, optical disk drive, solid-state thumb drive, and the like. The storage device may also be other similar means for loading computer programs or other instructions into the computer or processor.

As used herein, the term “computer” or “module” or “processor” or “workstation” may include any processor-based or microprocessor-based system including systems using microcontrollers, reduced instruction set computers (RISC), ASICs, logic circuits, and any other circuit or processor capable of executing the functions described herein. The above examples are exemplary only, and are thus not intended to limit in any way the definition and/or meaning of these terms.

The computer or processor executes a set of instructions that are stored in one or more storage elements, in order to process input data. The storage elements may also store data or other information as desired or needed. The storage element may be in the form of an information source or a physical memory element within a processing machine.

The set of instructions of an ultrasound system including those controlling the acquisition, processing, and transmission of ultrasound images as described above may include various commands that instruct a computer or processor as a processing machine to perform specific operations such as the methods and processes of the various embodiments of the invention. The set of instructions may be in the form of a software program. The software may be in various forms such as system software or application software and which may be embodied as a tangible and non-transitory computer readable medium. Further, the software may be in the form of a collection of separate programs or modules, a program module within a larger program or a portion of a program module. The software also may include modular programming in the form of object-oriented programming. The processing of input data by the processing machine may be in response to operator commands, or in response to results of previous processing, or in response to a request made by another processing machine.

Furthermore, the limitations of the following claims are not written in means-plus-function format and are not intended to be interpreted based on 35 U.S.C. 112, sixth paragraph, unless and until such claim limitations expressly use the phrase “means for” followed by a statement of function devoid of further structure.

1. An ultrasonic diagnostic imaging system for performing CW Doppler analysis comprising:
a linear array transducer probe having a given number of transducer elements arranged in a flat row or a curved arc, some of the transducer elements being multiplexed to a single multiplexer terminal and others of the transducer elements not being multiplexed;
a system beamformer having a number of beamformer channels which is less that the given number of transducer elements of the linear array transducer;
a B mode processor coupled to the system beamformer;
a Doppler processor coupled to the system beamformer;
a display processor coupled to the B mode processor and the Doppler processor and adapted to produce a B mode image or a CW spectral Doppler image for display; and
a display coupled to the display processor,
wherein the B mode image is produced from signals produced by the linear array probe using multiplexed transducer elements and the CW spectral Doppler image is produced from signals produced by the linear array probe using elements that are not multiplexed.

2. The ultrasonic diagnostic imaging system of claim 1, wherein the transducer elements which are multiplexed are multiplexed in pairs to one or more multiplexers, each of which has a common terminal coupled to a beamformer channel.

3. The ultrasonic diagnostic imaging system of claim 2, wherein the system beamformer is coupled to receive signals from transducer elements which are multiplexed during production of a B mode image.

4. The ultrasonic diagnostic imaging system of claim 3, wherein the system beamformer is also coupled to receive signals from transducer elements which are not multiplexed during production of a B mode image.

5. The ultrasonic diagnostic imaging system of claim 2, wherein the system beamformer is coupled to transmit signals and receive signals only using transducer elements which are not multiplexed during CW Doppler analysis.

6. The ultrasonic diagnostic imaging system of claim 5, wherein the system beamformer is coupled to a first group of transducer elements comprising a transmit aperture for transmission during CW analysis, and is coupled to a second group of transducer elements comprising a receive aperture for reception during CW analysis.

7. The ultrasonic diagnostic imaging system of claim 6, wherein the transmit aperture is located on one side of the linear array and the receive aperture is located on the other side of the linear array.

8. The ultrasonic diagnostic imaging system of claim 7, wherein the transmit and receive apertures are selectably changeable depending upon the side of the linear array where a target is located.

9. The ultrasonic diagnostic imaging system of claim 8, wherein the transmit aperture is selectably located on the side of the linear array where the target is located.

10. The ultrasonic diagnostic imaging system of claim 2, wherein the linear array transducer is a curved array comprising at least 160 transducer elements and the system beamformer comprises 128 beamformer channels.

11. A method for performing CW Doppler analysis comprising:
scanning a target anatomy with a linear array transducer probe to acquire a B mode image, the linear array being either curved or flat and comprising a plurality of transducer elements which are multiplexed and a plurality of transducer elements which are not multiplexed, multiplexed and unmultiplexed elements being coupled to a beamformer with fewer channels than the number of transducer elements of the array transducer;
adjusting a Doppler line to intersect a target in the B mode image;
adjusting a sample volume cursor over the target in the B mode image; and
starting CW Doppler signal acquisition using elements of the array transducer which are not multiplexed.

12. The method of claim 11, wherein starting CW Doppler signal acquisition using elements of the linear array transducer which are not multiplexed further comprises performing CW Doppler signal analysis using only elements of the linear array transducer which are not multiplexed.

13. The method of claim 12 wherein starting CW Doppler signal acquisition further comprises using a first group of transducer elements of the linear array transducer for transmission and using a second group of transducer elements of the linear array transducer for reception.

14. The method of claim 13, wherein the group of transducer elements used for transmission comprise a transmit aperture located on one side of the linear array, and the group of transducer elements used for reception comprise a receive aperture located on the other side of the linear array.

15. The method of claim 14, further comprising adjusting an angle correction cursor over the B mode image prior to starting CW Doppler signal acquisition.
